(19) United States
(12) Patent Application Publication
(10) Pub. No.: US 2023/0406802 A1
(43) Pub. Date: Dec. 21, 2023

(54) HYDROFORMYLATION REACTION PROCESSES

(71) Applicant: Dow Technology Investments LLC, Midland, MI (US)

(72) Inventors: Jason F. Giles, Missouri City, TX (US); Pritish M. Kamat, Pearland, TX (US); Glenn A. Miller, South Charleston, WV (US); George R. Phillips, South Charleston, WV (US); Chi-Wei Tsang, Lake Jackson, TX (US); Quan Yuan, Sugar Land, TX (US)

(21) Appl. No.: 18/251,256

(22) PCT Filed: Nov. 18, 2021
(86) PCT No.: PCT/US2021/059813

§ 371 Date: May 1, 2023

Related U.S. Application Data
(60) Provisional application No. 63/128,909, filed on Dec. 22, 2020.

(51) Int. Cl. C07C 45/50; B01J 19/26; B01J 10/00; B01J 19/00

(52) U.S. Cl. CPC C07C 45/505; B01J 19/26; B01J 10/002; B01J 19/0066

(57) ABSTRACT

The present invention relates to hydroformylation reaction processes. In one aspect, a hydroformylation reaction process comprises (a) contacting an olefin, hydrogen, and carbon monoxide in the presence of a homogeneous catalyst in a reactor to provide a reaction fluid, wherein the reactor comprises one or more reaction zones; (b) removing a portion of the reaction fluid from a first reaction zone; (c) passing at least a portion of the removed reaction fluid through a shear mixing apparatus to produce bubbles in the portion of the removed reaction fluid, wherein at least a portion of hydrogen and carbon monoxide provided to the reactor is introduced through the shear mixing apparatus; and (d) returning the removed reaction fluid to the first reaction zone through one or more nozzles wherein the returning reaction fluid exiting each nozzle is a jet, wherein the mixing energy density provided to the reactor by the jets is greater than or equal to 500 Watts/m³.

### FIELD

The present invention relates generally to hydroformylation reaction processes.

### INTRODUCTION

Hydroformylation is the reaction of olefins with H₂ and CO in the presence of an organophosphorous ligand-modified homogeneous rhodium catalyst to produce aldehydes according to the following equation:

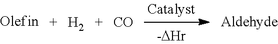

Typically the hydroformylation reaction is carried out in the liquid phase where syngas (a gaseous mixture of H₂ and CO) is sparged into the reaction fluid containing the liquid olefin, product aldehyde, heavies, and the homogeneous rhodium/ligand catalyst.

In order for the reaction to occur, H₂ and CO must be dissolved in the reaction fluid—hence effective gas/liquid mixing is critical to both initiate and maintain the hydroformylation reaction.

In addition, the heat generated by the exothermic hydroformylation reaction must be removed and the reactor temperature controlled at desired reaction conditions. This is typically achieved by internal cooling coils or recirculating the reaction fluid through an external heat exchanger and returning the cooled reaction fluid to the reactor or both.

Furthermore, under the same conditions as the above hydroformylation reaction, the resulting aldehyde may react further and be hydrogenated in situ to give the corresponding alcohol, and the hydroformylation under aminating conditions can be considered a variant of a hydroformylation reaction.

Another secondary catalytic activity of some hydroformylation catalysts is the hydrogenation or isomerization of double bonds, for example of olefins having internal double bonds, to saturated hydrocarbons or α-olefins, and vice versa. It is important to avoid these secondary reactions of the hydroformylation catalysts to establish and maintain specific hydroformylation reaction conditions in the reactor. Even small deviations from the process parameters can lead to the formation of considerable amounts of undesired secondary products, and maintaining virtually identical process parameters over the volume of the entire reaction liquid volume in the hydroformylation reactor may therefore be of considerable importance. Additionally, volumes within the reactor without sufficiently dispersed or dissolved syngas do not contribute to the reaction or productivity of the reactor. In addition, many hydrolysable catalysts exhibit catalyst degradation in the absence of syngas at reaction temperatures such that these regions of low dispersed or dissolved syngas will contribute towards decline in catalyst performance. Alternatively, many rhodium phosphine catalysts exhibit degradation in high CO environments such that regions of excessively high dissolved syngas concentrations should also be avoided. Thus, a highly dispersed (as determined by high gas hold-up or gas fraction) and uniform gas mixing is the most desirable outcome. In general, in the hydroformylation of olefins with organophosphorous ligand-modified homogeneous rhodium catalysts, it is advantageous to establish an optimum concentration of hydrogen and carbon monoxide dissolved in the liquid reaction medium.

The concentration of dissolved carbon monoxide (CO) in the reaction liquid is especially important and is a key hydroformylation reactor control variable. While the dissolved CO concentration in the reaction liquid cannot be measured directly, it is typically monitored and approximated using an on-line analyzer to measure the CO partial pressure in the vapor space of the reactor which is presumed to be in equilibrium with the reaction liquid phase. This approximation improves if the reaction fluid in the reactor is more uniformly mixed and better approximates the completely backed-mixed reaction mixture such as in the classical CSTR model.

Reactors with multiple zones such as described in U.S. Pat. No. 5,728,893 are preferred to achieve high conversion. However, in a reactor with more than one reaction zone, measuring the CO partial pressure of the headspace may only give an indication of the CO concentration in the top zone and not necessarily the CO concentration in the lower reactions zone(s). This becomes more important when the top reaction zone is not a back-mixed reactor. In the latter case, it is even more important that the feeds to the non-back-mixed reaction zone be as uniform as possible to achieve as uniform and/or predictable a CO distribution as possible.

The hydrocarbon (paraffin) formation reaction, the formation of high-boiling condensates of the aldehydes (i.e., high boilers or “heavies”), as well as the degradation rate of the organophosphorous-rhodium based catalyst are also influenced by the reaction temperature. For back-mixed reactors, it is important to avoid the formation of gradients with respect to the reaction temperature and the concentration of dissolved CO within the volume of the reaction liquid present in the reactor; in other words, it is important for close to identical operating conditions to be established and maintained over the total liquid volume. Thus, it is preferred to avoid non-homogenous distribution of reagents and temperature within a reaction zone. However, it is known that other non-back-mixed reactors such as plug flow and bubble reactors will have gradients thus making these reactors more difficult to control in this manner.

The means to feed the syngas and ensure a good distribution has been recognized previously. Academic articles have focused on agitation speed for example and the technology disclosed in PCT Publication No. WO2018/236823 for back-mixed reactors without a mechanical agitator teaches that good distribution of the syngas is critical for good reactivity and reactor performance.

It would therefore be desirable to have a hydroformylation reactor design and preferably a multi-zoned hydroformylation reactor design that provides highly dispersed and uniform syngas and temperature distribution in a reactor and establishes good initial syngas distribution without the use of a mechanical agitator.

### SUMMARY

The present invention generally relates to hydroformylation reaction processes where aldehydes are prepared by reacting olefins in the liquid phase with carbon monoxide and hydrogen gases. A portion of these gases are dispersed in the form of gas bubbles in a reaction liquid and another portion are dissolved in the reaction liquid, in the presence of a catalyst at elevated temperatures of 50° C. to 145° C. and at pressures of 1 to 100 bar various embodiments. Embodiments of the present invention can advantageously provide thorough gas-liquid mixing of a reaction fluid in a reactor without the use of a mechanical agitator.

It has been found that high velocity fluid flow can be utilized to (1) introduce the syngas as a well distributed flow of fine bubbles and (2) uniformly distribute the bubbles to mix the entire reaction zone by imparting momentum and shear into the reaction liquid to not only mix the reactor contents but also to disperse the syngas bubbles. Despite not being at the top of the reactor as in prior venturi gas/liquid mixing reactor designs, in some embodiments of the present invention, the overall reactor fluid can achieve remarkably uniform temperature and gas-liquid mixing as evidenced by higher and more uniform gas fraction or gas loading and constant and uniform temperature in the reactor. In addition, the uniformly mixed, fine bubbles facilitate introduction of the process fluid into non-backmixed reaction zones such as bubble columns or plug flow reactors which is difficult with venturi-style reactor designs.

In one aspect, a hydroformylation reaction process comprises (a) contacting an olefin with gaseous hydrogen, and carbon monoxide in the presence of a homogeneous catalyst in a reactor to provide a reaction fluid, wherein the reactor comprises one or more reaction zones; (b) removing a portion of the reaction fluid from a first reaction zone; (c) passing at least a portion of the removed reaction fluid through a shear mixing apparatus to produce bubbles in the portion of the removed reaction fluid, wherein at least a portion of hydrogen and carbon monoxide provided to the reactor is introduced through the shear mixing apparatus; and (d) returning the removed reaction fluid to the first reaction zone through one or more nozzles wherein the returning reaction fluid exiting each nozzle is a jet, wherein the mixing energy density provided to the reactor by the jets meets the following formula:

$$\frac{( {\sum }_{i = 1}^{i = N} \frac{1}{2} {ρ}_{i} {Q}_{i}^{3} / {A}_{i}^{2} )}{V} \geq 500 Watts / {m}^{3}$$

wherein V is the volume of the reaction fluid in the first reaction zone (in m³), N is the total number of jets being returned to the first reaction zone such that each jet is uniquely identified using natural numbers from i=1 to i=N (in increments of 1), ρᵢ is average density of the reaction fluid at the nozzle port being returned to the first reaction zone through the i^{th} jet (in kg/m³), Qᵢ is volumetric flow rate (in m³/s) of the reaction fluid being returned to the first reaction zone through the i^{th} jet, and Aᵢ is cross-sectional area (in m²) of the i^{th} nozzle through which the reaction fluid flows at the location where the reaction fluid exits the nozzle and enters the first reaction zone.

These and other embodiments are described in more detail in the Detailed Description.

### BRIEF DESCRIPTION OF THE FIGURES

FIG. 1 is a schematic illustrating an example of a hydroformylation reactor and related equipment that can be used for a hydroformylation reaction process according to one embodiment of the present invention.

FIG. 2 is a schematic illustrating the angles at which nozzles may be oriented in the reactor and other parameters according to some embodiments of the present invention.

FIG. 3 illustrates two embodiments of shear mixing apparatuses that can be used in some embodiments of the present invention, with “G” representing gas entering the shear mixing apparatus and “L” representing liquid entering the shear mixing apparatus.

FIG. 4 is a series of figures illustrating different positions of the nozzles within a reactor, different positions of one or more donut baffles relative to the jets, and the angles of jets exiting the nozzles according to some embodiments of the present invention.

FIG. 5 shows gas volume fraction contours for Comparative Example A and Inventive Examples 1 and 2.

FIG. 6 shows average value of mass transfer coefficient (kLa) contours for Comparative Example A and Inventive Examples 1 and 2.

### DETAILED DESCRIPTION

A hydroformylation process generally comprises contacting CO, H₂, and at least one olefin under hydroformylation conditions sufficient to form at least one aldehyde product in the presence of a catalyst comprising, as components, a transition metal and an organophosphorous ligand. Optional process components include an amine and/or water.

All references to the Periodic Table of the Elements and the various groups therein are to the version published in the CRC Handbook of Chemistry and Physics, 72nd Ed. (1991-1992) CRC Press, at page I-10.

Unless stated to the contrary or implicit from the context, all parts and percentages are based on weight and all test methods are current as of the filing date of this application. As used herein, the term “ppmw” means parts per million by weight. For purposes of United States patent practice, the contents of any referenced patent, patent application or publication are incorporated by reference in their entirety (or its equivalent US version is so incorporated by reference) especially with respect to the disclosure of definitions (to the extent not inconsistent with any definitions specifically provided in this disclosure) and general knowledge in the art.

As used herein, “a,” “an,” “the,” “at least one,” and “one or more” are used interchangeably. The terms “comprises,” “includes,” and variations thereof do not have a limiting meaning where these terms appear in the description and claims. Thus, for example, an aqueous composition that includes particles of “a” hydrophobic polymer can be interpreted to mean that the composition includes particles of “one or more” hydrophobic polymers.

Also herein, the recitations of numerical ranges by endpoints include all numbers subsumed in that range (e.g., 1 to 5 includes 1, 1.5, 2, 2.75, 3, 3.80, 4, 5, etc.). For the purposes of the invention, it is to be understood, consistent with what one of ordinary skill in the art would understand, that a numerical range is intended to include and support all possible subranges that are included in that range. For example, the range from 1 to 100 is intended to convey from 1.01 to 100, from 1 to 99.99, from 1.01 to 99.99, from 40 to 60, from 1 to 55, etc.

As used herein, the term “hydroformylation” is contemplated to include, but is not limited to, all permissible asymmetric and non-asymmetric hydroformylation processes that involve converting one or more substituted or unsubstituted olefinic compounds or a reaction mixture comprising one or more substituted or unsubstituted olefinic compounds to one or more substituted or unsubstituted aldehydes or a reaction mixture comprising one or more substituted or unsubstituted aldehydes.

The terms “reaction fluid,” “reaction medium” and “catalyst solution” are used interchangeably herein, and may include, but are not limited to, a mixture comprising: (a) a metal-organophosphorous ligand complex catalyst, (b) free organophosphorous ligand, (c) aldehyde product formed in the reaction, (d) unreacted reactants (e.g., hydrogen, carbon monoxide, olefin), (e) a solvent for said metal-organophosphorous ligand complex catalyst and said free organophosphorous ligand, and, optionally, (f) one or more ligand degradation products such as oxides and phosphorus acidic compounds formed in the reaction (which may be homogeneous or heterogeneous, and these compounds include those adhered to process equipment surfaces). It should be understand that the reaction fluid can be a mixture of gas and liquid. For example, the reaction fluid can include gas bubbles (e.g., hydrogen and/or CO and/or inerts) entrained within a liquid or gases (e.g. hydrogen and/or CO and/or inerts) dissolved in the liquid. The reaction fluid can encompass, but is not limited to, (a) a fluid in a reaction zone, (b) a fluid stream on its way to a separation zone, (c) a fluid in a separation zone, (d) a recycle stream, (e) a fluid withdrawn from a reaction zone or separation zone, (f) a withdrawn fluid being treated with an aqueous buffer solution, (g) a treated fluid returned to a reaction zone or separation zone, (h) a fluid on its way to an external cooler, (i) a fluid in an external cooler, (j) a fluid being returned to a reaction zone from an external cooler, and (k) ligand decomposition products and their salts.

As used herein, the term “first reaction zone” in a multiple reaction zone reactor or reaction train refers to the reaction zone into which the bulk of the catalyst is introduced (e.g., recycled catalyst or catalyst-containing reaction fluid from an upstream reactor not part of this invention). The “second reaction zone” follows the first reaction zone in that the bulk of the catalyst flows from the first reaction zone to the second reaction zone, and so on. The advantages of this type of reaction scheme is described in U.S. Pat. No. 5,728,893. For the purposes of this invention, the term “first reaction zone” is related to the reaction zone wherein most of the olefin, syngas, and catalyst are introduced to the reactor. The majority of the reaction fluid leaving this first reaction zone is then transported to the “second reaction zone” through perforated plates without intermediary piping. In this context, “first” and “second” are related to the path followed by the bulk of the catalyst in this reactor recognizing that there may be reaction zones prior to this reactor body which are not included in this invention.

The present invention generally relates to hydroformylation reaction processes where aldehydes are prepared by reacting olefins in the liquid phase with carbon monoxide and hydrogen gases. Embodiments of the present invention advantageously disperse at least a portion of the carbon monoxide and/or hydrogen gases in the form of small gas bubbles in the reaction fluid. In some embodiments, the processes of the present invention can advantageously provide thorough gas-liquid mixing of the reaction fluid without the use of a mechanical agitator.

In one embodiment, a hydroformylation process of the present invention comprises (a) contacting an olefin, hydrogen, and carbon monoxide in the presence of a homogeneous catalyst in a reactor to provide a reaction fluid, wherein the reactor comprises one or more reaction zones; (b) removing a portion of the reaction fluid from a first reaction zone; (c) passing at least a portion of the removed reaction fluid through a shear mixing apparatus to produce bubbles in the portion of the removed reaction fluid, wherein at least a portion of hydrogen and carbon monoxide provided to the reactor is introduced through the shear mixing apparatus; and (d) returning the removed reaction fluid to the first reaction zone through one or more nozzles wherein the returning reaction fluid exiting each nozzle is a jet, wherein the mixing energy density provided to the reactor by the jets meets the following formula:

$$\frac{( {\sum }_{i = 1}^{i = N} \frac{1}{2} {ρ}_{i} {Q}_{i}^{3} / {A}_{i}^{2} )}{V} \geq 500 Watts / {m}^{3}$$

wherein V is the volume of the reaction fluid in the first reaction zone (in m³), N is the total number of jets being returned to the first reaction zone such that each jet is uniquely identified using natural numbers from i=1 to i=N (in increments of 1), ρᵢ is average density of the reaction fluid being at the nozzle port returned to the first reaction zone through the i^{th} jet (in kg/m³), Qᵢ is volumetric flow rate (in m³/s) of the reaction fluid being returned to the first reaction zone through the i^{th} jet, and Aᵢ is cross-sectional area (in m²) of the i^{th} nozzle through which the reaction fluid flows at the location where the reaction fluid exits the nozzle and enters the first reaction zone. In some embodiments, in addition to hydrogen and carbon monoxide being provided to the reactor through the shear mixing apparatus, inert gases (e.g., methane, CO₂, argon, nitrogen, etc.) may also be present in the syngas provided to the reactor through the shear mixing apparatus. In some embodiments, the average bubble size of the bubbles generated by the shear mixing apparatus is between 10 nanometers and 3,000 microns. In some embodiments, the average bubble size of the bubbles generated by the shear mixing apparatus is between 100 microns and 800 microns.

The flow rate of the reaction fluid through the shear mixing apparatus can be important to facilitate adequate mixing of the reaction fluid. In one embodiment, the flow rate of the reaction fluid through the shear mixing apparatus meets the following:

q_{SM}>525(μₒ/ρₒ)P_{SM}

wherein q_{SM} is the flow rate (m³/s) of the reaction fluid entering the shear mixing apparatus, wherein ρₒ is the density (kg/m³) of the reaction fluid prior to entering the shear mixing apparatus, wherein μₒ is the viscosity (Pa-s) of the reaction fluid prior to entering the shear mixing apparatus, and wherein P_{SM} is the smallest wetted perimeter of the cross-section for liquid flow inside the shear mixing apparatus.

In some embodiments, the removed reaction fluid is returned to the first reaction fluid through at least two nozzles, wherein each nozzle is oriented such that an angle of the nozzle relative to a horizontal plane (alpha) is between +750 and −75°, and wherein alpha, an angle of the nozzle relative to a vertical plane passing through the center of the reactor (beta), and a distance from the vertical plane passing through center of the reactor when beta is zero (phi) are all not zero.

In some embodiments, hydrogen and carbon monoxide are provided as syngas, and at least 20% of syngas provided to the first reaction zone passes through the shear mixing apparatus prior to entering the first reaction zone.

In some embodiments, at least a portion of the syngas is introduced in the cylindrical reactor through a sparger at a height that is less than 50% of the reaction fluid-filled height of the first reaction zone.

In some embodiments, the reactor comprises a horizontally oriented ring baffle attached to an inside wall of the reactor, wherein the ring baffle is positioned at a height that is less than 90% of the height of the liquid reaction fluid within the first reaction zone, wherein the solid portion of the ring baffle extends from 5 to 30% of the diameter of the reactor.

In some embodiments, an agitator is positioned in the reactor. In some embodiments, the agitator is not operating. In some embodiments, the agitator and the returning reaction fluid provide the mixing energy density in the cylindrical reactor.

The reactor is vertically-oriented in some embodiments.

The reactor, in some embodiments, further comprises a second reaction zone, wherein the reaction fluid flows from the first reaction zone to the second reaction zone without piping. In some further embodiments, the first reaction zone and the second reaction zone are separated by a perforated plate. The reactor, in some embodiments, further comprises a third reaction zone, wherein the reaction fluid flows from the second reaction zone to the third reaction zone without piping. In some further embodiments, the second reaction zone and third reaction zone are separated by a perforated plate.

In some embodiments, the reactor comprises a product outlet nozzle positioned in a lower portion of the reactor, as well as means for preventing gas entrainment positioned in a bottom volume of the reactor.

The hydroformylation process of the present invention comprises contacting an olefin, hydrogen, and carbon monoxide in the presence of a homogeneous catalyst in a reactor to provide a reaction fluid, wherein the reactor comprises one or more reaction zones

Hydrogen and carbon monoxide may be obtained from any suitable source, including petroleum cracking and refinery operations. Syngas mixtures are a preferred source of hydrogen and CO. Syngas (from synthesis gas) is the name given to a gas mixture that contains varying amounts of CO and H₂. Production methods are well known. Hydrogen and CO typically are the main components of syngas, but syngas may contain CO₂ and inert gases such as N₂ and Ar. The molar ratio of H₂ to CO varies greatly but generally ranges from 1:100 to 100:1 and usually between 1:10 and 10:1. Syngas is commercially available and is often used as a fuel source or as an intermediate for the production of other chemicals. The H₂:CO molar ratio for chemical production is often between 3:1 and 1:3 and usually is targeted to be between about 1:2 and 2:1 for most hydroformylation applications.

A solvent advantageously is employed in typical embodiments of the hydroformylation process. Any suitable solvent that does not unduly interfere with the hydroformylation process can be used. By way of illustration, suitable solvents for rhodium catalyzed hydroformylation processes include those disclosed, for example, in U.S. Pat. Nos. 3,527,809; 4,148,830; 5,312,996; and 5,929,289. Non-limiting examples of suitable solvents include saturated hydrocarbons (alkanes), aromatic hydrocarbons, water, ethers, aldehydes, ketones, nitriles, alcohols, esters, and aldehyde condensation products. Specific examples of solvents include: tetraglyme, pentanes, cyclohexane, heptanes, benzene, xylene, toluene, diethyl ether, tetrahydrofuran, butyraldehyde, and benzonitrile. The organic solvent may also contain dissolved water up to the saturation limit. Illustrative preferred solvents include ketones (e.g. acetone and methylethyl ketone), esters (e.g. ethyl acetate, di-2-ethylhexyl phthalate, 2,2,4-trimethyl-1,3-pentanediol monoisobutyrate), hydrocarbons (e.g. toluene), nitrohydrocarbons (e.g. nitrobenzene), ethers (e.g. tetrahydrofuran (THF)) and sulfolane. In rhodium catalyzed hydroformylation processes, it may be preferred to employ, as a primary solvent, aldehyde compounds corresponding to the aldehyde products desired to be produced and/or higher boiling aldehyde liquid condensation by-products, for example, as might be produced in situ during the hydroformylation process, as described, for example, in U.S. Pat. Nos. 4,148,830 and 4,247,486. The primary solvent will normally eventually comprise both aldehyde products and higher boiling aldehyde liquid condensation by-products (“heavies”), due to the nature of the continuous process. The amount of solvent is not especially critical and need only be sufficient to provide the reaction medium with the desired amount of transition metal concentration. Typically, the amount of solvent ranges from about 5 percent to about 95 percent by weight, based on the total weight of the reaction fluid. Mixtures of solvents may be employed.

Embodiments of the present invention are applicable to improving any conventional continuous mixed gas/liquid phase CSTR rhodium-phosphorus complex catalyzed hydroformylation process for producing aldehydes, which process is conducted in the presence of free organophosphorus ligand. Such hydroformylation processes (also called “oxo” processes) and the conditions thereof are well known in the art as illustrated, e.g., by the continuous liquid recycle process of U.S. Pat. No. 4,148,830, and phosphite-based processes of U.S. Pat. Nos. 4,599,206 and 4,668,651. Also included are processes such as described in U.S. Pat. Nos. 5,932,772 and 5,952,530. Such hydroformylation processes in general involve the production of aldehydes by reacting an olefinic compound with hydrogen and carbon monoxide gas in a liquid reaction medium which contains a soluble rhodium-organophosphorus complex catalyst, free organophosphorus ligand and higher boiling aldehyde condensation by-products. In general, rhodium metal concentrations in the range of from about 10 ppm to about 1000 ppm by weight, calculated as free metal, should be sufficient for most hydroformylation processes. In some processes, about 10 to 700 ppm by weight of rhodium is employed, often, from 25 to 500 ppm by weight of rhodium, calculated as free metal.

Accordingly, as in the case of the rhodium-organophosphorus complex catalyst, any conventional organophosphorus ligand can be employed as the free ligand and such ligands, as well as methods for their preparation, are well known in the art. A wide variety of organophosphorous ligands can be employed with the present invention. Examples include, but are not limited to, phosphines, phosphites, phosphino-phosphites, bisphosphites, phosphonites, bisphosphonites, phosphinites, phosphoramidites, phosphino-phosphoramidites, bisphosphoramidites, fluorophosphites, and the like. The ligands may include chelate structures and/or may contain multiple P(III) moieties such as polyphosphites, polyphosphoramidites, etc. and mixed P(III) moieties such as phosphite-phosphoramidites, flurophosphite-phosphites, and the like. Of course, mixtures of such ligands can also be employed, if desired. Thus, the hydroformylation process of this invention may be carried out in any excess amount of free phosphorus ligand, e.g., at least 0.01 mole of free phosphorus ligand per mole of rhodium metal present in the reaction medium. The amount of free organophosphorus ligand employed, in general, merely depends upon the aldehyde product desired, and the olefin and complex catalyst employed. Accordingly, amounts of free phosphorus ligand present in the reaction medium ranging from about 0.01 to about 300 or more per mole of rhodium (measured as the free metal) present should be suitable for most purposes. For example, in general, large amounts of free triarylphosphine ligand, e.g., triphenylphosphine, such as more than 50 moles or, in some cases, more than 100 moles of free ligand per mole of rhodium have been employed to achieve satisfactory catalytic activity and/or catalyst stabilization, while other phosphorus ligands, e.g., alkylarylphosphines and cycloalkylarylphosphines may help provide acceptable catalyst stability and reactivity without unduly retarding the conversion rates of certain olefins to aldehydes when the amount of free ligand present in the reaction medium is as little as 1 to 100 and, in some cases, 15 to 60 moles per mole of rhodium present. In addition, other phosphorus ligands, e.g., phosphines, sulfonated phosphines, phosphites, diorganophosphites, bisphosphites, phosphoramidites, phosphonites, fluorophosphites, may help provide acceptable catalyst stability and reactivity without unduly retarding the conversion rates of certain olefins to aldehydes when the amount of free ligand present in the reaction medium is as little as 0.01 to 100 and, in some cases, 0.01 to 4 moles per mole of rhodium present.

More particularly, illustrative rhodium-phosphorus complex catalysts and illustrative free phosphorus ligands include, e.g., those disclosed in U.S. Pat. Nos. 3,527,809; 4,148,830; 4,247,486; 4,283,562; 4,400,548; 4,482,749; European Patent Application Publication Nos. 96,986; 96,987 and 96,988 (all published Dec. 28, 1983); and PCT Publication No. WO 80/01690 (published Aug. 21, 1980). Among the more preferred ligands and complex catalysts that may be mentioned are, e.g., the triphenylphosphine ligand and rhodium-triphenylphosphine complex catalysts of U.S. Pat. Nos. 3,527,809 and 4,148,830 and 4,247,486; the alkylphenylphosphine and cycloalkylphenylphosphine ligands, and rhodium-alkylphenylphosphine and rhodium-cycloalkylphenylphosphine complex catalysts of U.S. Pat. No. 4,283,562; and the diorganophosphite ligands and rhodium-diorganophosphite complex catalysts of U.S. Pat. Nos. 4,599,206 and 4,668,651.

As further noted above, the hydroformylation reaction is typically carried out in the presence of higher boiling aldehyde condensation by-products. It is the nature of such continuous hydroformylation reactions employable herein to produce such higher boiling aldehyde by-products (e.g., dimers, trimers and tetramers) in situ during the hydroformylation process as explained more fully, e.g., in U.S. Pat. Nos. 4,148,830 and 4,247,486. Such aldehyde by-products provide an excellent carrier for the liquid catalyst recycle process. For example, initially the hydroformylation reaction can be effected in the absence or in the presence of small amounts of higher boiling aldehyde condensation by-products as a solvent for the rhodium complex catalyst, or the reaction can be conducted in the presence of upwards of 70 weight percent, or even as much as 90 weight percent, and more of such condensation by-products, based on the total liquid reaction medium. In general, ratios of aldehyde to higher boiling aldehyde condensation by-products within the range of from about 0.5:1 to about 20:1 by weight should be sufficient for most purposes. Likewise it is to be understood that minor amounts of other conventional organic co-solvents may be present if desired.

While the hydroformylation reaction conditions may vary over wide limits, as discussed above, in general it is more preferred that the process be operated at a total gas pressure of hydrogen, carbon monoxide and olefinic unsaturated starting compound of less than about 3100 kiloPascals (kPa) and more preferably less than about 2415 kPa. The minimum total pressure of the reactants is not particularly critical and is limited mainly only by the amount of reactants necessary to obtain a desired rate of reaction. More specifically, the carbon monoxide partial pressure of the hydroformylation reaction process of this invention can be from about 1 to 830 kPa and, in some cases, from about 20 to 620 kPa, while the hydrogen partial pressure can be from about 30 to 1100 kPa and, in some cases, from about 65 to 700 kPa. In general, the H₂:CO molar ratio of gaseous hydrogen to carbon monoxide may range from about 1:10 to 100:1 or higher, about 1:1.4 to about 50:1 in some cases.

Further, as noted above, the hydroformylation reaction process of this invention may be conducted at a reaction temperature from about 50° C. to about 145° C. However, in general, hydroformylation reactions at reaction temperatures of about 60° C. to about 120° C., or about 65° C. to about 115° C., are typical.

Of course it is to be understood that the particular manner in which the hydroformylation reaction is carried out and particular hydroformylation reaction conditions employed are not narrowly critical to the subject invention and may be varied widely and tailored to meet individual needs and produce the particular aldehyde product desired.

External cooling loops (pumped circulation of the reactor contents through an external heat exchanger (cooler)) are typically used for highly exothermic hydroformylation reactions such as for lower carbon olefins (C2 to C5) since internal cooling coils alone often lack sufficient heat removal capacity (limited heat transfer area per coil volume). In addition, internal cooling coils displace internal reactor volume making the reactor size larger for a given production rate. However, in some embodiments, at least one internal cooling coil is positioned inside the reactor typically the first reaction zone. Such internal cooling coil(s) can be in addition to an external cooling loop, in some embodiments. In a preferred embodiment, the liquid process fluid used to generate the jets (either separately or with the high shear microbubble generator modifications) is passed through a heat exchanger (preferably before the microbubble generator) prior to being reintroduced back to the same reaction zone. The flows of the cooled process fluid can be varied for optimal temperature control of the reaction zone as taught, for example, in U.S. Pat. No. 9,670,122 (FIG. 3 in particular).

Preferred examples of the olefins that can be used as reactants in the present invention include ethylene, propylene, butene, 1-hexene, 1-octene, 1-nonene, 1-decene, 1-undecene, 1-tridecene, 1-tetradecene, 1-pentadecene, 1-hexadecene, 1-heptadecene, 1-octadecene, 1-nonadecene, 1-eicosene, 2-butene, 2-methyl propene, 2-pentene, 2-hexene, 2-heptene, 2-ethyl hexene, 2-octene, styrene, 3-phenyl-1-propene, 1,4-hexadiene, 1,7-octadiene, 3-cyclohexyl-1-butene, allyl acetate, allyl butyrate, methyl methacrylate, vinyl methyl ether, vinyl ethyl ether, allyl ethyl ether, n-propyl-7-octenoate, 3-butenenitrile, 5-hexenamide, 4-methyl styrene, 4-isopropyl styrene, and the like. Mixtures of isomers (e.g., butene raffinates) can also be employed. The resulting aldehydes products may be subjected to hydrogenation, and thus converted into corresponding alcohols which may be used as a solvent or for the preparation of plasticizer, or may undergo other subsequent reactions such as aldol condensation to higher aldehydes, oxidation to the corresponding acids, or esterification to produce the corresponding acetic, propionic, or acrylic esters.

The olefin starting material is introduced to the reactor by any convenient technique either as a gas (optionally with the incoming syngas feed), as a liquid in the reactor, or as part of a recirculation loop prior to entry into the reactor. One particularly useful method is to use a separate olefin sparger next to or below the jets or the optional syngas sparger (discussed below) to introduce the olefin and syngas feeds in close proximity to each other.

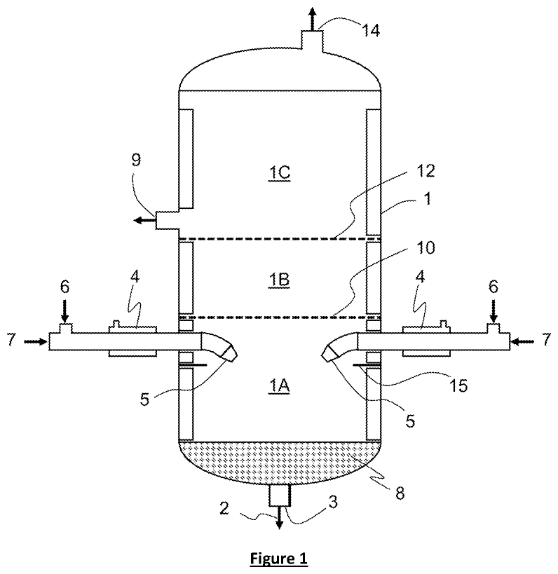

To help illustrate operation of some embodiments of the hydroformylation reaction process of the present invention, reference will now be made to FIG. 1. FIG. 1 illustrates a non-limiting example of a cylindrical reactor 1 that can be used to implement a hydroformylation reaction process according to one embodiment of the present invention. The reactor 1 includes a reaction fluid that is a mixture of olefin, hydrogen, carbon monoxide, homogeneous catalyst, aldehyde product, solvent, and other components. The reactor has three reaction zones 1A,1B,1C. A portion of the reaction fluid is removed from the first reaction zone 1A through outlet 3 in the bottom of the reactor. At least a portion of the removed reaction fluid is passed through two shear mixing apparatuses 4 where fresh or recycled syngas (with or without inerts) is introduced as shown in FIG. 3a or FIG. 3b to generate gas bubbles in the portion of the removed reaction fluid. The removed reaction fluid is returned to the first reaction zone 1A through two nozzles 5. The nozzles and their orientation are discussed further below. The removed reaction fluid being returned to the first reaction zone 1A through the nozzles 5 forming one or more liquid jets of returning reaction fluid which impart momentum and gas/liquid mixing in the bulk reactor fluid. The shear mixing apparatuses are such as those described in U.S. Pat. No. 5,845,993, which is hereby incorporated by reference.

With regard to the reaction fluid removed from the bottom of the reactor 1 via stream 2, crude product and a catalyst mixture can be removed from stream 2 via a product-catalyst separation zone (not shown). This stream 2 may also be passed through a heat removal process as well such that the returning process fluid is cooled which in turn will cool the reaction zone.

As used herein, the terms “shear mixing apparatus,” “high shear mixing apparatus,” “microbubble generator,” and “high shear microbubble generator” are used interchangeably and refer to a device that can generate gas bubbles having an average size of 3,000 microns or less in a fluid. A key feature and advantage of the shear mixing apparatus that can be used in embodiments of the present invention is that it is constructed entirely of static piping components (e.g., does not include moving parts or require a mechanical seal which eliminates the need for maintenance and eliminates a potential leak/failure point), and thus increases inherent safety, mechanical reliability, reduced environmental releases, and plant on-stream time. Examples of shear mixing apparatuses that can be used in embodiments of the present invention are described in U.S. Pat. No. 5,845,993, which is hereby incorporated by reference. In general, the shear mixing apparatus comprises a pressurized gas conduit or chamber in contact with a single (or multiple) turbulent liquid stream(s) separated by a perforated surface. The gas enters into the liquid stream(s) through the perforations driven by the shear stress created by the liquid flow. Two typical embodiments of such shear mixing apparatuses are shown in FIG. 3. For example, in one embodiment (FIG. 3a), the shear mixing apparatus has an inner channel carrying a liquid stream (L). This is fitted with an outer concentric jacket connected to a pressurized gas inlet (G). A portion of the inner channel enveloped by the outer jacket is perforated with a number of perforations. These perforations are where the gas (G) from the outer jacket enters the liquid (L) flow in the inner channel in the form of a gas-in-liquid dispersion composed of small bubbles. In the present invention, the liquid (L) is at least a portion of the removed reaction fluid that is to be returned to the first reaction zone, and the gas (G) is syngas.

In some embodiments, a portion of the syngas can also be introduced to the first reaction zone through a conventional sparger ring (such as disclosed in PCT Publication No. WO2018/236823), in addition to syngas introduced through the shear mixing apparatus(es). In other embodiments, the only source of syngas provided to the first reaction zone is through the shear mixing apparatus(es).

In embodiments of the present invention, the mixing energy being introduced to the first reaction zone without a traditional sparger ring is different from PCT Publication No. WO2018/236823 because the bubbles are generated by the shear mixing apparatus(es). The momentum generated by the flow through the shear mixing apparatus(es) needs to distribute the bubbles evenly throughout the reaction fluid starting at the exits of the nozzles. The majority of the momentum from the jets leaving the nozzles need not reach to the bottom of the first reaction zone, in some embodiments where traditional sparger rings are used, and only distribute the bubbles throughout the first reaction zone. To achieve suitable mixing and gas dispersion, there are several considerations related to the reactor and nozzle design that need to be addressed as discussed further below.

As set forth in the mixing energy density formula below, we have found that if the mixing energy density (power delivered per unit volume) provided by the jets exceeds 500 W/m³, excellent results will be achieved. In the absence of such mixing energy, the lower (or zero) turbulence in the reaction fluid results in larger diameter gas bubbles sizes which quickly rise up to the gas/liquid interface due to increased buoyancy forces and disengage from the liquid, resulting in lower gas holdup in the reactor. Generating and maintaining small bubbles are important to producing a uniform reaction fluid which will give better gas/liquid mixing, gas hold-up and more reproducible reactor performance. Smaller bubbles allow for maximum gas hold-up and maximize mass transfer area between the bubbles and the liquid for dissolving the syngas (optimized gas volume/surface ratio). Conversely, very small bubbles may be captured in the stream lines of the liquid near an outlet nozzle (for example, to the external recirculation pump/heat exchanger or in the reactor product outlet) which may negatively impact downstream equipment so a key feature of the invention is the ability to consistently generate bubbles in the appropriate size range.

Referring again to FIG. 1, reaction fluid is removed from the bottom of the reactor 1 via outlet 3 is returned to the reactor via two or more nozzles 5 optionally terminated with diverter plates or restricting nozzles (discussed below). The two or more nozzles 5, in some embodiments, can be oriented in symmetrical pairs, symmetrical triads or other symmetrical arrangements.

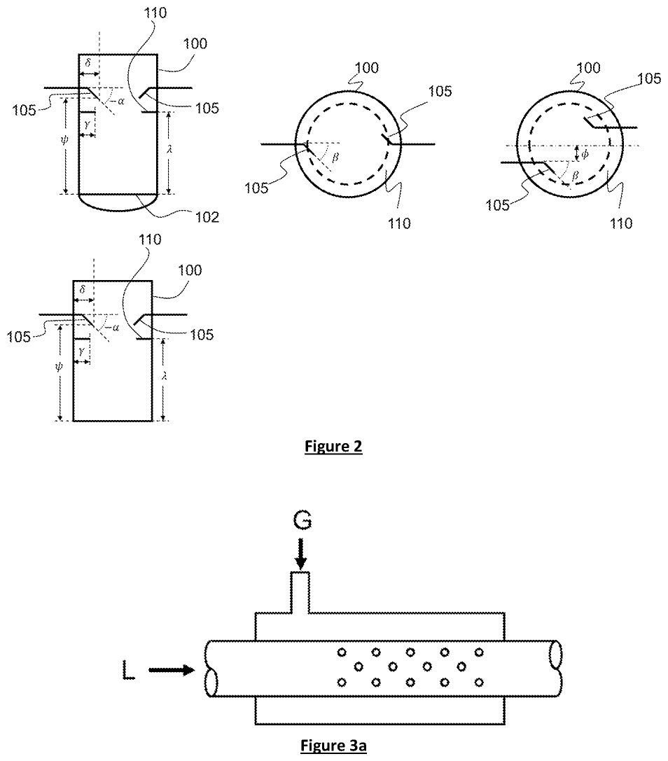

The nozzles 5 can be oriented so as to direct the liquid jets in a downward or upward direction or both. In some embodiments, the nozzles can be oriented such that the liquid jets are not directed toward a center vertical axis of the reactor 1 (e.g., not toward the reactor center line). It is preferred that the liquid jets are not oriented in a strictly horizontal or strictly vertical direction or directly toward the vertical axis or center of the reactor. Orientation of the nozzles is discussed further below in connection with FIG. 2.

In some embodiments, multiple sets of symmetrical nozzles can be positioned at different nozzle orientations (radial position) and/or different heights in the reactor 1. In some embodiments, various liquid feeds (e.g., liquid olefin feed, a liquid catalyst stream an upstream reactor, a liquid catalyst stream from a product-catalyst separation zone, etc.) can be provided to the reactor 1 through the shear mixing apparatuses 4. In some embodiments, one or more of such feeds can be combined with the returning removed reaction fluid and provided to the reactor 1 through at least one shear mixing apparatus. If liquid feed is from an upstream reactor, there may be some syngas present but this represents a minor amount of syngas compared to the syngas introduced by the shear mixing apparatuses 4. In the embodiment shown in FIG. 1, fresh liquid olefin feed 6 is combined with returning reaction fluid 7 and provided to the reactor 1 via the shear mixing apparatus.

The returning removed reaction fluid exits each nozzle 5 as a jet. As used herein, the terms “jets,” “directed jets,” and “directed streams” are used interchangeably and are described in PCT Publication No. WO2018/23623 except that the syngas is being delivered by one or more shear mixing apparatuses rather than sparger rings. The jets may be the output of one or more shear mixing apparatuses or separate streams designed specifically for mixing the first reaction zone (separately or in conjunction with the shear mixing apparatuses).

The jets provide a downward and countercurrent flow to counterbalance the natural buoyancy of the bubbles and maintain entrainment of the bubbles in the liquid circulating throughout the back-mixed reactor, which results in a more uniform distribution of the syngas bubbles throughout the back-mixed liquid phase. As the syngas dissolves and reacts, the bubbles will shrink which further helps in maintaining their distribution within the back-mixed liquid phase and in promoting good gas mass transfer into the liquid phase. As this uniformly mixed liquid reaction fluid moves up into a non-agitated reaction zone across a permeable physical barrier such as a perforated divider plate (discussed below), it will react in a controlled manner without the need for external mixing energy to be supplied in some embodiments.

The jets of returning reaction fluid provide mixing energy density to the reaction fluid in order to adequately mix the reactants in the reaction fluid to facilitate reaction.

In some embodiments, the jets provide sufficient mixing energy density such that an agitator or other mechanical source of mechanical mixing energy is not needed. The jets provide mixing energy density that meets the following formula:

$$\frac{( {\sum }_{i = 1}^{i = N} \frac{1}{2} {ρ}_{i} {Q}_{i}^{3} / {A}_{i}^{2} )}{V} \geq 500 Watts / {m}^{3}$$

wherein V is the volume of the reaction fluid in the first reaction zone (in m³), N is the total number of jets being returned to the first reaction zone such that each jet is uniquely identified using natural numbers from i=1 to i=N (in increments of 1), ρᵢ is average density of the reaction fluid at the nozzle port being returned to the first reaction zone through the i^{th} jet (in kg/m³), Qᵢ is volumetric flow rate (in m³/s) of the reaction fluid being returned to the first reaction zone through the i^{th} jet, and Aᵢ is cross-sectional area (in m²) of the i^{th} nozzle through which the reaction fluid flows at the location where the reaction fluid exits the nozzle and enters the first reaction zone. For clarity, V (the volume of the reaction fluid in the first reaction zone in m³) refers to the gas-filled liquid level as the process is being run (as opposed to the degassed liquid volume). This volume (V) can be determined by known methods such as sonar level indicators or take-off nozzles. Similarly, ρᵢ can be readily calculated by the relative flows of reaction fluid and syngas being fed to the shear mixing apparatus. The average density of the reaction fluid (ρ) at the nozzle port being returned to the first reaction zone through the i^{th} jet (in kg/m³), the volumetric flow rate (Qᵢ) (in m³/s) of the reaction fluid being returned to the first reaction zone through the i^{th} jet, and the cross-sectional area (Aᵢ) (in m²) of the i^{th} nozzle through which the reaction fluid flows can be measured or determined using techniques known to those of ordinary skill in the art based on the teachings herein. By providing a mixing density energy (as defined in the above formula) of 500 Watts/m³ or more, the jets are believed to provide adequate mixing to the first reaction zone. In other words, in some embodiments, the jets can sufficiently mix without the need of a conventional mechanical agitator.

The flow rate of the reaction fluid through the shear mixing apparatus can also be important to ensure that adequate mixing energy is provided to the first reaction zone. Thus, in some embodiments, the flow rate of the reaction fluid through the shear mixing apparatus meets the following:

q_{SM}>525(μₒ/ρₒ)P_{SM}

wherein q_{SM} is the flow rate (m³/s) of the reaction fluid entering the shear mixing apparatus, wherein ρₒ is the density (kg/m³) of the reaction fluid prior to entering the shear mixing apparatus, wherein μₒ is the viscosity (Pa-s) of the reaction fluid prior to entering the shear mixing apparatus, and wherein P_{SM} is the smallest wetted perimeter of the cross-section for liquid flow inside the shear mixing apparatus. The flow rate (m³/s) of the reaction fluid entering the shear mixing apparatus (q_{SM}), the density (kg/m³) of the reaction fluid prior to entering the shear mixing apparatus (ρₒ), and the viscosity (Pa-s) of the reaction fluid prior to entering the shear mixing apparatus (μₒ) can be measured using techniques known to those of ordinary skill in the art based on the teachings herein. The smallest wetted perimeter of the cross-section for liquid flow inside the shear mixing apparatus (P_{SM}) can be determined as follows. For a conventional tube transporting the reaction fluid through the shear mixing apparatus, P_{SM} is pi multiplied by the inner diameter of the tube (P_{SM}=ID_{tube}). In some cases, there may be an inner tube as well with the reaction fluid flowing in the annular region between an outer wall of the inner tube and an inner wall of the outer tube. In this situation, P_{SM} is ρᵢ multiplied by the sum of the outer diameter of the inner tube and the inner diameter of the outer tube (P_{SM}=π(OD_{inner tube}+ID_{outer tube})).

In one embodiment, all of the jets are from shear mixing apparatuses. In other embodiments, some jets are solely for imparting mixing energy density while others are from one or more shear mixing apparatuses. In another embodiment, a multi-zoned reactor has shear mixing apparatus jet loops in multiple reaction zones within the reactor wherein each jet loop recirculates fluid taken from the same zone as it was withdrawn. In another embodiment, a multi-zoned reactor can be configured so as to remove reaction fluid from a first reaction zone and return the reaction fluid into a second reaction zone as a jet via a shear mixing apparatus. In a further embodiment, all the zones within the reactor body have jets from high shear mixing apparatuses. In a preferred embodiment, the second reaction zone is not a back-mixed reactor but chosen from a bubble column reactor, plug flow reactor, a piston flow reactor, a gas- or bubble-lift (tubular) reactor, a packed bed reactor, or a venturi-style reactor. Examples of non-back-mixed reactors include U.S. Pat. Nos. 5,367,106, 5,105,018, 7,405,329, and 8,143,468.

The position and orientation of nozzles within the reactor is important, especially when two or more nozzles are provided. For example, one should generally avoid positioning two nozzles such that the jets exiting the nozzles would be oriented directly at each other. FIG. 2 provides a rough schematic of a side view and two top views of a cylindrical reactor 100 to illustrate the position and orientation of nozzles 105 according to some embodiments of the present invention. FIG. 2 also shows a donut baffle 110 (discussed further below) positioned beneath the nozzles 105 in the reactor 100. Alpha (α) is the angle of the nozzles relative to a horizontal plane. In some embodiments, with a horizontal angle being 0°, α can range between 75° (angled upward) and −75° (angled downward). In some embodiments, with a horizontal angle being 0°, α can range between 45° (angled upward) and −60° (angled downward). Beta (β) is the angle that the nozzles are oriented left or right relative to a center line. In some embodiments, with an orientation where the nozzle is directed straight across the reactor being 0°, β is generally between 5° and 90° (the nozzle facing clockwise as viewed from the top of the reactor) or between −5° and −90° (the nozzle facing counterclockwise as viewed from the top of the reactor). β should only be between −5° and 5° if phi (ϕ) is greater than 0°. Phi (ϕ) is the distance that the nozzles are off-set from a center-line of the reactor when viewed from the top. ϕ should be no more than 50% of the cross-sectional diameter of the reactor. In some embodiments where at least two nozzles return the removed reaction fluid to the reactor, each nozzle is oriented such that an angle of the nozzle relative to a horizontal plane (alpha (α)) is between +75° and −75°, and wherein alpha (α), an angle of the nozzle relative to a vertical plane passing through the center of the reactor (beta (β)), and a distance from the vertical plane passing through center of the reactor when beta is zero (phi (ϕ)) are all not zero.

It should be understood that the flow of returning reaction fluid will not be in a single line in some embodiments, but that the majority of the reaction fluid returning to the reactor in a single nozzle will be within a relatively narrow range of α and β values. For the purposes of this application, when the terms “vertical” and “horizontal” are used in connection with the flow of returning reaction fluid at a fluid diverter, the terms can be understood using angles α and β, respectively. That is, a “vertical stream” or “vertical jet” is oriented up and/or down at a not equal to zero but β essentially zero. A “horizontal stream” or “horizontal jet” is oriented going left and/or right at a essentially zero but β not equal to zero. The term “directed streams” generally refers to streams that have both α and β not equal to zero. The “directed streams” may include a stream from a shear mixing apparatus or other streams that are returning but not pass through a shear mixing apparatus.

Referring still to FIG. 2, delta (δ) is the distance that a nozzle projects into the reactor from the reactor wall. δ is less than 50% of the diameter of the reactor in some embodiments. In some embodiments, δ is not greater than 50% of the radius of the cylindrical reactor. In some embodiments, δ is at least 10% of the radius of the cylindrical reactor. δ is from 10% to 45% of the radius of the cylindrical reactor in some embodiments. In some embodiments, the end of the flow diverter can be generally flush with the reactor wall such that δ is ˜0% of the radius of the cylindrical reactor.

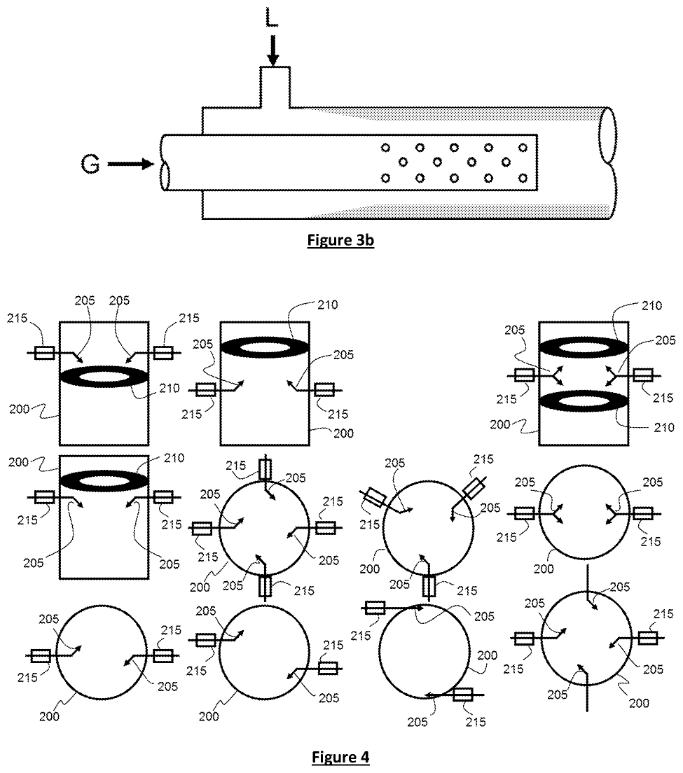

In some embodiments, additional sets of nozzles can be provided at the same or different heights as shown in FIG. 2 or at different angles (α and/or β). FIG. 4 is a series of figures illustrating different positions of the nozzles 205 within reactors 200, different positions of one or more donut baffles 210 relative to the jets (not labelled but represented by arrows exiting the ports of the nozzles 205), and the angles of jets exiting the nozzles 205 according to some embodiments of the present invention. Shear mixing apparatuses 215 are also shown, but not labelled on each of the illustrations in FIG. 4.

Psi (ψ) is the distance (as a percentage of the reaction fluid-filled height) at which the tip of a nozzle is located. As used herein, the “reaction fluid-filled height” refers to the height of the liquid in the reactor from the bottom of the reactor. As shown in FIG. 2, in embodiments where the reactor has a headspace in the bottom portion, all heights referenced as being measured from the bottom of the reactor are measured from a tangent line 102 across the reactor just above the headspace. If the reactor has a flat bottom, as also shown in FIG. 2, all heights referenced as being measured from the bottom of the reactor are measured from the physical bottom. ψ is less than 100% of the reaction fluid-filled height. In some embodiments, ψ is less than 90% of the reaction fluid-filled height. In some embodiments, ψ is from 75% to 80% of the reaction fluid-filled height.

Each shear mixing apparatus is designed to introduce syngas bubbles into the removed reaction fluid. Without being bound by theory, the high liquid velocity and thorough mixing with small initial bubble size provided by embodiments of the present invention minimize syngas bubble coalescence, promotes bubble size reduction by shearing, and gives an even distribution of gas/liquid and temperature throughout the reaction zone. The movement of small syngas bubbles due to their natural buoyancy is countered by the viscosity of the liquid and the turbulent flow of the liquid mass. Likewise, when a non-agitated reaction zone is above the first reaction zone, the natural buoyancy up to and across the permeable physical barrier such as a grid or perforated plate separating the two zones is countered by the viscosity of the liquid and the turbulent flow of the liquid mass. Excessively large bubbles will rise too rapidly thus resulting in low gas holdup and non-uniform distribution. In some embodiments, the average size of the bubbles generated by a shear mixing apparatus can be between 10 nanometers and 3,000 microns. In some embodiments, the average of the bubbles generated by a shear mixing apparatus is between 3 microns and 3,000 microns. In some embodiments, the average of the bubbles generated by a shear mixing apparatus is between 30 microns and 3,000 microns. In some embodiments, the average size of the bubbles generated by a shear mixing apparatus is between 100 microns and 800 microns.

The manner in which the reaction fluid is returned impacts the effectiveness of the mixing energy provided. In some embodiments, the reaction fluid can be returned using pipes with one or more flow diverter plate(s) installed on the end of a section of pipe that is then inserted through and attached to the recirculation return nozzle(s) of the reactor. In some embodiments, the reaction fluid is returned using nozzles or flow orifices positioned at the end of a section of pipe that is then inserted through and attached to the recirculation return nozzle(s) of the reactor as discussed further below. In each instance, the resulting liquid jet(s) velocity is a function of the flow area of the nozzles or orifices, or the flow area created between the flow diverter plate(s) and the inside wall of the pipe, and the mass flow rate and density of the returning reaction fluid. The combination of flow area and flow rate results in a jet of reaction fluid inside the reactor that imparts momentum and induces gas/liquid and liquid/liquid mixing of the bulk fluid in the reactor. Further, the returning reaction fluid is divided and directed in a plurality of directions.

The term “flow diverter” is used herein to encompass both nozzles and diverter plates positioned in reactor recirculation return pipes. In either case, the flow diverters direct the flow of the returning reaction fluid. As discussed further below, the flow diverters direct the flow of the returning reaction fluid horizontally in some embodiments. In some embodiments, the flow diverters direct the flow of the returning reaction fluid vertically. The flow diverters direct the flow of the returning reaction fluid both horizontally and vertically in some embodiments. Flow diverters comprising flow diverter plates positioned in the end of pipes are described in more detail in PCT Publication No. WO2018/236823, which is hereby incorporated by reference.

Horizontal donut baffles over or under the nozzles are used in some embodiments to mitigate the flow or channeling effects within the reactor from the jets. The donut baffle is a flat, ring plate fixed to the reactor wall with a central opening, which serves to break up channeling flows along the reactor wall. Non-limiting examples of the placement of such horizontal donut baffles are shown in FIG. 1 (reference number 14), FIG. 2 (reference number 110) and FIG. 4 (reference number 210). As shown in FIG. 2, the donut baffle 110 extends a distance (γ) from the reactor wall. In some embodiments, the donut baffle extends a distance (γ) from the reactor wall that is 5% to 25% of the diameter of the reactor. The vertical location of the donut baffle within the reactor can also be important in some embodiments. As shown in FIG. 2, the donut baffle 110 is positioned at a certain height (λ) from the bottom of the reactor. In some embodiments, the donut baffle can be positioned at a height (λ) from the bottom of the reactor that is 90% or less of the reaction fluid-filled height. Other approaches may be used to minimize the potential for flow or channeling effects from the reaction fluid entering the reactor as jets (see, e.g., the position of the donut baffle 14 in FIG. 1 and of the donut baffles 210 in FIG. 4).

In some embodiments, such as the embodiment shown in FIG. 1, the reaction fluid is from the first reaction zone through a product outlet nozzle 3 at the bottom of the reactor. In such embodiments, the reactor can comprise means for preventing gas entrainment 8 positioned in a bottom volume of the reactor. Such means can in the form of an entrainment separator, a conical coalesce, one or more perforated plates, or a packed bed. A packed bed 8 is shown in FIG. 1. Such means for gas entrainment 8 may be particularly desirable when the jets are angled downward, but may not be needed if the recirculating pumps can tolerate small entrained gas bubbles.

In some embodiments, perforated divider plates can be positioned between reaction zones when a single reactor includes multiple reaction zones. For example, as shown in FIG. 1, the reaction fluid from the first reaction zone 1A passes up into the second reaction zone 1B through a perforated divider plate 10. The perforated divider plate 10 can help ensure that the reaction fluid moving up into the second reaction zone 1B is uniform and comprises a substantial amount of syngas for the continued reaction. This is particularly desirable for bubble reactors, plug flow reactors, and packed column reactors in that the reagents are very uniform and not diffusion limited.

In the embodiment shown in FIG. 1, a second perforated divider plate 12 separates the second reaction zone 1B from the third reaction zone 1C.

To be effective, the perforated divider plate holes should be evenly distributed so as to disperse the rising fluid evenly across the cross-section of the reactor. In plug-flow or packed bed column reactors, the perforations should direct flows to ensure each tube or column gets the same fluid flow. The design of perforated divider plates or trays are well known in the art. A typical perforated divider plate/tray should have 15-40% (preferably 20-30%) porosity with the perforations evenly distributed throughout the surface. The perforations may be uniform or have different diameters with equivalent hole diameters ranging typically from ⅛″ to 2″. The holes may be round, square, slots, or other shapes and may have additional features (e.g., counter-sunk, raised holes, etc.), but should not accumulate significant amounts of gas under the perforated divider plates. Wire mesh or similar rigidly supported materials may be used as alternatives to perforated divider plates in some embodiments.

Vertical baffles can be attached to the interior walls of the first reaction zone to provide further mixing and minimize rotational flow by shearing and lifting radial streamlines from the vessel wall.

Returning to FIG. 1, in the final reaction zone 1C, a reactor outlet 9 is present to convey the reaction fluid to the next reactor or to a product-catalyst separation zone (not shown).

In addition, an optional gas purge stream 14 from the reactor 1 can be vented, flared, sent to the plant fuel gas header or to another reactor in embodiments where multiple reactors are arranged in series. Analysis of this purge stream 14 can provide a convenient means to measure CO partial pressure in the top reaction zone for reaction control.

While not shown in FIG. 1, the system also includes other standard pieces of equipment such as pumps, heat exchangers, cooling coils, valves, level sensors, temperature sensors, and pressure sensors, which are easily recognized and implemented by those skilled in the art.

In some embodiments, the removed reaction fluid that is returned to the first reaction zone through the one or more nozzles can provide at least 50% of the total mixing energy density to the first reaction zone. The removed reaction fluid that is returned to the first reaction zone through the one or more nozzles, in some embodiments, can provide at least 85% of the total mixing energy density to the first reaction zone. In some embodiments, the returning reaction fluid can provide substantially all or 100% of the total mixing energy density to the first reaction zone. It should be understood that the total mixing energy density comprises mixing energy density provided by an operating agitator (if present), by the jets of returning reaction fluid, or any other source of mixing energy density, but does not include any de minimis mixing energy density that might be provided by the introduction of the syngas, olefin, or other reactant feed to the reactor. For example, there is some de minimis mixing energy density supplied by the hydraulic flow of liquid reaction fluid from the first reaction zone through the subsequent reaction zones (e.g., through permeated divider plates when present) which is also not included. In embodiments where the liquid jets produced by the returning reaction fluid provides substantially all or 100% of the mixing energy density, the reactor either does not include an agitator, or includes an agitator that is not in operation.

When an agitator is present and operating, the contribution of mixing energy density from the agitator can be calculated using the following formula:

P=N_{pg}×ρN³D⁵

where N_{pg} is the gassed power number for the impeller, ρ is density of the reaction fluid, N is the rotational speed of the agitator (rev/s), and D is the diameter of the impeller.

Surprisingly, it has been found that employing shear mixing apparatus(es) as described herein can enable the operation of a hydroformylation reactor without an agitator being operated while providing the same level of gas/liquid and liquid/liquid mixing of the reaction fluid. Providing an increase in flow of returning reaction fluid can enable stable operation without an operating agitator and facilitate superior gas dispersion into the liquidation reaction fluid. By providing adequate mixing in the reactor without use of an agitator, some embodiments of the present invention can advantageously permit continued operation of a reactor that does have an agitator if there are issues with an agitator motor, agitator seals, agitator shaft/impeller, steady bearing or similar agitator-related issues until such time as the unit can be shut down and repairs can be made thus avoiding unplanned loss of production. In other words, in a retrofit situation, some embodiments of the present invention can permit an existing agitator to not be operated and/or to be repaired while still operating the reactor. For new reactors, some embodiments of the present invention can advantageously eliminate the cost of an agitator as well as the need for agitator seals and steady bearings which require maintenance/replacement, and can eliminate seal leaks.

Some embodiments of the present invention will now be described in detail in the following Examples.

### EXAMPLES

In the present Examples, computational fluid dynamics (“CFD”) tools are used to evaluate the performance of three designs. Comparative Example A is representative of prior art technology in which a mechanical agitator is used. Inventive Examples 1 and 2 represent embodiments of the present invention utilizing a shear mixing apparatus without a mechanical agitator. The objective is to show the equivalence and/or improvement in terms of performance criteria of the inventive agitator-free designs (Inventive Example 1 and Inventive Example 2) over the conventional, mechanically agitated design (Comparative Example A). CFD is used here to evaluate performance in terms of: (a) mixing effectiveness (i.e., mixing time); (b) gas dispersion (i.e., uniformity of gas volume fraction and overall gas holdup); (c) degassing (i.e., volume % of gas in the bottom recirculation line; and (d) mass transfer (i.e., average value of the mass transfer coefficient (kLa) in the first reaction zone).

### Importance of Dispersing Syngas

It is important for several reasons to have highly and well dispersed syngas in the reactor. Since only the syngas that is dispersed and dissolved in the reaction fluid can react, it is critical that the syngas introduced to the reactor is quickly dispersed and dissolved into the reaction fluid rather than rising as bubbles to the vapor/liquid interface where it disengages and enters the vapor space of the reactor and is no longer available for reaction. Additionally, volumes within the reactor without dispersed or dissolved syngas are starved for a reactant, and thus do not contribute to the reaction or productivity of the reactor. Thus, a highly dispersed (high gas hold-up or gas fraction) and uniform gas mixing is the most desirable outcome.

### How Effectiveness is Evaluated in CFD

To assess the mixing characteristics of the present invention, it is convenient to examine the gas distributions from the CFD modeling to identify both the uniformity in gas distribution and the extent of gas loading. Commercial experience with well-agitated CSTR reactors have gas loading values in the 5-12% range. CFD modelling programs can be used to predict an overall or average gas loading value for the entire reactor volume but this may de-emphasize localized effects of areas with high or low gas loading and short residence time (e.g., pipe inlets/outlets, near agitator impellers, etc.).

Mixing Effectiveness: Mixing time θₘᵢₓ
For a well-mixed system, the mixing time θₘᵢₓ should typically be smaller than 10-20% of the average liquid residence time θᵣₑₛ. (See Paul, E. L., V. A. Atiemo-Obeng, and S. M. Kresta, eds. 2004. Handbook of Industrial Mixing: Science and Practice. John Wiley & Sons, Inc.)
In the present CFD simulations, mixing time θₘᵢₓ is evaluated for the first reaction zone in each Example.
The well-known tracer injection method is implemented. The simulation is first run without a tracer. Once steady state is achieved, a tracer is continuously injected at the fresh feed inlet and its concentration is tracked in the first reaction zone. At every simulation time step, the Coefficient of Variation (CoV) is evaluated as the volumetric standard deviation of the concentration over its volumetric mean.
Mixing time is defined as the flow time at which CoV reaches 5%.

Gas Dispersion: Uniformity of Gas Volume Fraction and Overall Gas Holdup.
To assess the mixing characteristics of the present invention, it is convenient to examine the gas distributions from the CFD modeling to identify both the uniformity in gas distribution and the extent of gas loading.
Commercial experience with well-agitated CSTR reactors have gas loading values in the 5-12% range.

Degassing: Volume % of Gas in the Bottom Recirculation Line
The bottom outlet of the reactor vessel typically leads to a centrifugal pump that is used to recirculate the fluid.
Syngas bubbles, if small enough, can be entrained into the outlet. If entrainment is large enough, the presence of the gas can damage the pump.
For safe pump operation, it is essential to keep the gas volume fraction in the bottom recirculation line below 3-5%.
In the CFD modeling, this volume fraction is tracked and this value is reported for each case.

Mass Transfer:
The overall effectiveness of a reacting gas-liquid system such as the hydroformylation system rests on how fast the syngas components (CO and H2) are transferred to the liquid phase.
The rate of mass transfer of syngas to the liquid phase in any reaction zone is directly proportional to the average value of the mass transfer coefficient (kLa) in that reaction zone.
Here CFD modeling is used to directly obtain the average value of the mass transfer coefficient (kLa) in the first reaction zone. A method well-documented in the literature is used for this purpose. See Gimbun, Reilly and Nagy “Modelling of mass transfer in gas-liquid stirred tanks agitated by Rushton turbine and CD-6 impeller: A scale-up study” Chemical engineering research and design 87 (2009) 437-451
For equivalent performance of two reactors conducting the same gas liquid reaction at the same operating and feed conditions, the overall volumetric kLa values must be equivalent. In general, higher kLa values are preferred.

### Operating Conditions and Parameters

For each of the Examples, the following operating conditions and parameters are used. The operating pressure is around 15 bar abs. The density of the liquid propylene is approximately 775 kg/m³, and the density of syngas is approximately 9.06 kg/m³, at this pressure. The feed flow rates of syngas and liquid propylene for each of the Examples are also given in Table 1. The viscosity of liquid propylene is taken to be 3.8×10⁻⁴ Pa·s, and the viscosity of syngas is taken to be 1.8×10⁻⁵ Pa·s. The gas-liquid surface tension between the syngas and the liquid propylene is taken to be 18 dynes/cm (0.018 N/m), in keeping with typical values for similar organics.

### Comparative Example A

The original reactor is a mechanically agitated tank having a diameter of 5.5 meters and a cylindrical section height of 10 meters capped at the top and bottom by two identical 2:1 semi-ellipsoidal heads. The volume of the tank is vertically divided into three reaction zones (numbered 1-3 from bottom to the top) by two horizontal baffles. The baffles are identical stainless steel plates having the same diameter as the tank and a single central orifice of diameter of 0.7 meter. Additionally, the tank is fitted with 4 identical vertical baffles along the reactor walls, spaced 90° apart.

The syngas is introduced using two identical ring spargers located in the first reaction zone (0.2 m above the bottom tangent line) and in the second reaction zone (0.2 m above the lower horizontal baffle). The agitator is a shaft fitted with three impellers: a standard gas-distribution turbine in the bottom compartment and two hydrofoils in the second and third reaction zones. The agitator operates at 89 rpm.

A degassing ring, concentric with the reactor body is attached to the bottom dished head around the bottom recirculation nozzle.

Table 1 summarizes the reactor dimensions and flow rates.

TABLE 1
Comparative Example A

| Dimensions and Flow Rates | Base case |
|---|---|
| Reactor diameter (m) | 5.5 |
| L/D | 2.32 |
| Recirculation inlet size | 12″ NB. |
| Recirculation outlet size | 16″ NB. |
| Straight (cylindrical) height of the reactor (m) | 10.0 |
| Recirculation nozzle height above bottom tangent line (m) | 2.0 |
| Length of recirculation nozzle inside reactor (m) (δ) | 0.825 |
| Liquid recirculation flow rate (kg/h) | 150138 |
| Syngas Gas feed flow rate (kg/h) - First Reaction Zone | 2485 |
| Syngas Gas feed flow rate (kg/h) - Second Reaction Zone | 266 |
| Olefin Liquid feed flow rate (kg/h) | 57445 |

### Inventive Example 1

The reactor dimensions (diameter and L/D) are identical to Comparative Example A. The agitator is absent and the mixing and gas dispersion is instead carried out using the recirculation jets entering the first reaction zone. In addition, the following other modifications are made over the Comparative Example A:
1. The liquid recirculation flow rate is boosted by a factor of 16.
2. Gas Introduction and Bubble size: The sparger rings are removed. Syngas is now introduced directly into the recirculation stream using two shear mixing apparatuses, each located just upstream of a recirculation inlet nozzle. These shear mixing apparatuses are designed to introduce gas into the reactor at a mean bubble diameter of 300 microns. Details of the shear mixing apparatuses are provided in the Shear Mixing Apparatus portion at the end of the Examples section.
3. Horizontal Baffles: The horizontal baffles from the Comparative Example A design are replaced by stainless steel perforated plates. These plates have a 20% open area to allow the two-phase (gas-liquid) reaction fluid to pass vertically upwards from the bottom to middle to top compartment.
4. Nozzles: Wedge inserts from the recirculation inlet nozzles are removed. Each recirculation inlet nozzle is fitted with a curved section at the end such that
   a. The gas-liquid jet enters at a vertical angle (α) of 20 degrees (downward along reactor central axis), and an azimuthal angle (β) of 30 degrees (counter-clockwise about reactor central axis).
   b. The nozzle diameter is reduced at the end to 7″ nominal size using a standard conical reducer (12″×7″).
   c. The nozzle opening where the two-phase jet enters the first reaction zone is located at a height (ψ) of 2.52 m above the bottom tangent line of the reactor and 0.45 m radially inwards from the inner wall (δ) of the reactor vessel.
5. Degassing Internals: The degassing ring is removed. In its place, a packed bed is installed having a void fraction of 36% and a total height of 1.375 m.
6. Internals: A donut baffle is added to the first reaction zone to prevent channeling of the gas to the second reaction zone. The donut baffle is placed 2 m above the bottom tangent line and has a width of 0.59 m.
7. Bottom Recirculation Nozzle: The nozzle size is raised from the original size of 16″ to 22″ to reduce liquid velocity exiting the reactor.

Table 2 summarizes the various dimensions and other parameters for Inventive Example 1.

TABLE 2
Inventive Example 1

| | Inventive |
|---|---|
| Dimensions | Example 1 |
| Reactor diameter (m) | 5.5 |
| L/D | 2.32 |
| Recirculation inlet size | 12″ NB. |
| Recirculation outlet size | 16″ NB. |
| Straight (cylindrical) height of the reactor (m) | 10.0 |
| Recirculation nozzle height above bottom | 2.0 |
| tangent line (m) | |
| Length of recirculation nozzle inside reactor (m) (δ) | 0.45 |
| Liquid recirculation flow rate (kg/h) | 2,402,073 (same |
| | as R1 with 2 pumps) |
| Syngas Gas feed flow rate (kg/h) - First | 2751 |
| Reaction Zone | |
| Syngas Gas feed flow rate (kg/h) - Second | 0 |
| Reaction Zone | |
| Olefin Liquid feed flow rate (kg/h) | 57445 |

As previously discussed, FIG. 2 defines various parameters to characterize the orientation and position of nozzles within the reactor. For Inventive Examples 1 and 2, Table 3 provides the values of these parameters.

TABLE 3

| | | | Ratio as expressed |
|---|---|---|---|
| | | | in range (Optimum |
| Parameter | Value | | values/ratios) |
| Height of reaction zone (H) | 3.24 | m | — |
| Vessel diameter (T) | 5.5 | m | — |
| α | −20 | degrees | — |
| β | 30 | degrees | — |
| δ | 0.45 | m | 8.2% of T |
| γ | 0.59 | m | 10.7% of T |
| λ | 2.0 m (above | | 61.7% of H |
| | bottom tan line) | | |
| ϕ | 0 | m | 0% of T |
| ψ | 2.52 | m | 77.8% of H |

### Inventive Example 2

Inventive Example 2 is the same as Inventive Example 1 except for the following modifications:
1. Degassing Internals: The packed bed is removed. In its place, a stack of three horizontal perforated plates is used. The open area fractions are: 30% (top plate), 20% (middle plate), 15% (bottom plate). The gap between plates is 0.25 m.

### Results

The results of the CFD modeling are shown in Tables 4A and 4B3.

TABLE 4A

| | | | | | | | Total P/V |
|---|---|---|---|---|---|---|---|
| | Agitator | | Degassing | | | | in First |
| | in First | | Equipment | Recirculation | Inlet | Jet | Reaction |
| | Reaction | Gas | at Bottom | Flow Rate | Nozzle | Velocity | Zone |
| | Zone? | Introduction | Outlet | (kg/h) | size | (m/s) | (kW/m³) |
| Comparative | Y | Ring Spargers | Degassing | 150,138 | 12″ with | 1.4 | 3.04 |
| Example A | | | Ring | | wedge | | |
| | | | | | inserts | | |
| Inventive | N | Shear Mixing | Packed Bed | 2,402,073 | 7″ (no | 18.3 | 1.17 |
| Example 1 | | Apparatuses | | | wedge | | |
| (Packed bed) | | | | | inserts) | | |
| Inventive | N | Shear Mixing | Stack of 3 | 2,402,073 | 7″ (no | 18.3 | 1.17 |
| Example 2 | | Apparatuses | perforated | | wedge | | |
| (Perforated | | | plates | | inserts) | | |
| plates) | | | | | | | |

TABLE 4B

| | | | Average | | | | |
|---|---|---|---|---|---|---|---|
| | | | Liquid | | | | Average |
| | | | residence | Mixing | | | kLa in |
| | | Average | time | Time in | | | First |
| | | Bubble | in first | First | | Vol % gas in | Reaction |
| | Gas | Size | reaction | Reaction | Gas | Recirculation | Zone |
| | Introduction | (mm) | zone (s) | Zone(s) | Holdup | line | (s⁻¹) |
| Comparative | Ring Spargers | 15 | 4739 | 300 | 10% | 0% | 0.22 |
| Example A | | | | | uniform | | |
| Inventive | Shear Mixing | 0.30 | 4739 | 340 | 8% | <1% | 0.33 |
| Example 1 | Apparatuses | | | | uniform | | |
| (Packed bed) | | | | | | | |
| Inventive | Shear Mixing | 0.30 | 4739 | 610 | 7.2% | <1% | 0.34 |
| Example 2 | Apparatuses | | | | uniform | | |
| (Perforated | | | | | | | |
| plates) | | | | | | | |

As shown in Table 4, Inventive Examples 1 and 2 have equivalent performance (e.g., mixing time, kLa and vol % gas in recirculation line) relative to Comparative Example A despite not having a mechanical agitator. Inventive Examples 1 and 2 also have significantly lower power consumption (see Ply).

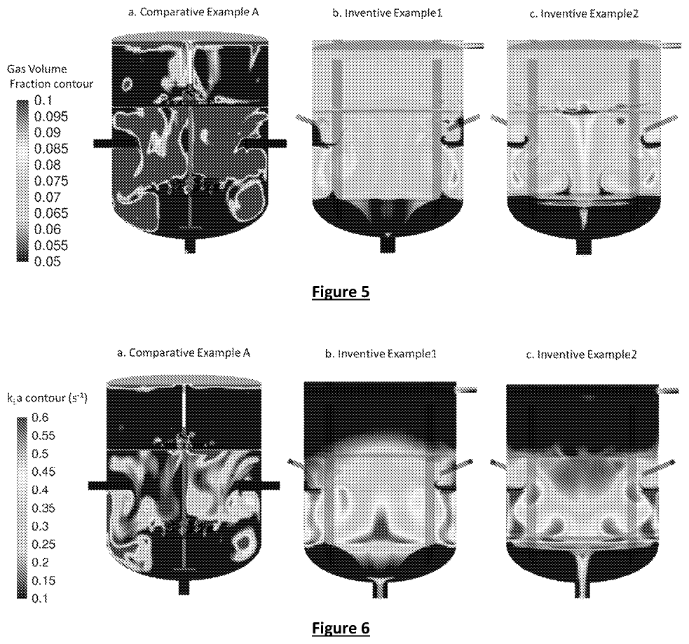

FIGS. 5 and 6 provide gas volume fraction contours and kLa contours for Comparative Example A and Inventive Examples 1 and 2. As shown in FIG. 5, the Inventive Examples have gas volume fractions that are very uniform.

### Shear Mixing Apparatus

The shear mixing apparatuses used in Inventive Examples 1 and 2 are of a type as described in U.S. Pat. No. 5,845,993. Each apparatus consists of a pressurized gas conduit or chamber in contact with a single (or multiple) turbulent liquid stream(s) separated by a perforated surface. The gas enters into the liquid stream(s) through the perforations driven by the shear stress created by the liquid flow.

For Inventive Examples 1 and 2, and as shown in FIG. 3a, the shear mixing apparatus is composed of an inner channel carrying a liquid stream. This is fitted with an outer concentric jacket connected to a pressurized gas inlet. A portion of the inner channel enveloped by the outer jacket is perforated with a number of perforations. These perforations are where the gas from the outer jacket enters the liquid flow in the inner channel in the form of a gas-in-liquid dispersion composed of small bubbles. In Inventive Examples 1 and 2, the liquid is reaction fluid withdrawn from the reactor, and the gas is syngas.

The shear mixing apparatus is configured so as to provide an average bubble size of 300 microns. The dimensions and flow rates in the shear mixing apparatus to provide this average bubble size are provided in Table 5.

TABLE 5

| Dimensions | |
|---|---|
| Inner channel (liquid) size | 12″ Sch. 40 |
| Outer jacket (gas) size | 14″ Sch. 40 |
| Length of the shear mixer element (in) | 24 |
| Number of holes | 10 |
| Hole diameter (in.) | ¼ |
| Gas-side pressure drop (psid) | 5.95 |
| Liquid-side pressure drop (psid) | 0.34 |
| Mean bubble size (μm) | 326 |
| Liquid flow rate through inner channel (gpm) | 2096 |
| Gas flow rate (kg/h) | 1375.5 |
| Gas-line inlet pressure (bar a) | 16-20 |

1. A hydroformylation reaction process, the process comprising:
(a) contacting an olefin, hydrogen, and carbon monoxide in the presence of a homogeneous catalyst in a reactor to provide a reaction fluid, wherein the reactor comprises one or more reaction zones;
(b) removing a portion of the reaction fluid from a first reaction zone;
(c) passing at least a portion of the removed reaction fluid through a shear mixing apparatus to produce bubbles in the portion of the removed reaction fluid, wherein at least a portion of hydrogen and carbon monoxide provided to the reactor is introduced through the shear mixing apparatus; and
(d) returning the removed reaction fluid to the first reaction zone through one or more nozzles wherein the returning reaction fluid exiting each nozzle is a jet, wherein the mixing energy density provided to the reactor by the jets meets the following formula:
$$\frac{( {\sum }_{i = 1}^{i = N} \frac{1}{2} {ρ}_{i} {Q}_{i}^{3} / {A}_{i}^{2} )}{V} \geq 500 Watts / {m}^{3}$$
wherein V is the volume of the reaction fluid in the first reaction zone (in m³), N is the total number of jets being returned to the first reaction zone such that each jet is uniquely identified using natural numbers from i=1 to i=N (in increments of 1), ρᵢ is average density of the reaction fluid at the nozzle port being returned to the first reaction zone through the i^{th} jet (in kg/m³), Qᵢ is volumetric flow rate (in m³/s) of the reaction fluid being returned to the first reaction zone through the i^{th} jet, and Aᵢ is cross-sectional area (in m²) of the i^{th} nozzle through which the reaction fluid flows at the location where the reaction fluid exits the nozzle and enters the first reaction zone.

2. The process of claim 1, wherein the flow rate of the reaction fluid through the shear mixing apparatus meets the following:
q_{SM}>525(μₒ/ρₒ)P_{SM}
wherein q_{SM} is the flow rate (m³/s) of the reaction fluid entering the shear mixing apparatus, wherein ρₒ is the density (kg/m³) of the reaction fluid prior to entering the shear mixing apparatus, wherein μₒ is the viscosity (Pa-s) of the reaction fluid prior to entering the shear mixing apparatus, and wherein P_{SM} is the smallest wetted perimeter of the cross-section for liquid flow inside the shear mixing apparatus.

3. The process of claim 1, wherein at least two nozzles return the removed reaction fluid to the reactor, wherein each nozzle is oriented such that an angle of the nozzle relative to a horizontal plane (alpha) is between +75° and −75°, and wherein alpha, an angle of the nozzle relative to a vertical plane passing through the center of the reactor (beta), and a distance from the vertical plane passing through center of the reactor when beta is zero (phi) are all not zero.

4. The process of claim 1, wherein hydrogen and carbon monoxide are provided as syngas, and wherein at least 20% of syngas provided to the first reaction zone passes through the shear mixing apparatus prior to entering the first reaction zone.

5. The process of claim 1, wherein hydrogen and carbon monoxide are provided as syngas, and wherein at least a portion of the syngas is introduced in the cylindrical reactor through a sparger at a height that is less than 50% of the reaction fluid-filled height of the first reaction zone.

6. The process of claim 1, wherein the reactor comprises a horizontally oriented ring baffle attached to an inside wall of the reactor, wherein the ring baffle is positioned at a height that is less than 90% of the height of the liquid reaction fluid within the first reaction zone, wherein the solid portion of the ring baffle extends from 5 to 30% of the diameter of the reactor.

7. The process of claim 1, further comprising an agitator positioned in the cylindrical reactor.

8. The process of claim 7, wherein the agitator and the returning reaction fluid provide the mixing energy density in the cylindrical reactor.

9. The process of claim 7, wherein the agitator is not operating.

10. The process of claim 1, wherein the reactor is vertically-oriented.

11. The process of claim 1, wherein the reactor further comprises a second reaction zone, wherein the reaction fluid flows from the first reaction zone to the second reaction zone without piping.

12. The process of claim 11, wherein the first reaction zone and the second reaction zone are separated by a perforated plate.

13. The process of claim 11, wherein the reactor further comprises a third reaction zone, wherein the reaction fluid flows from the second reaction zone to the third reaction zone without piping.

14. The process of claim 13, wherein the second reaction zone and third reaction zone are separated by a perforated plate.

15. The process of claim 1, wherein the average bubble size of the bubbles generated by the shear mixing apparatus is between 10 nanometers and 3,000 microns.

16. The process of claim 1, wherein the reactor comprises a product outlet nozzle positioned in a lower portion of the reactor, and wherein the reactor comprises means for preventing gas entrainment positioned in a bottom volume of the reactor.
